(19) United States
(12) Patent Application Publication
(10) Pub. No.: US 2021/0016069 A1
(43) Pub. Date: Jan. 21, 2021

(54) APPLICATORS FOR TREATING VAGINAL DRYNESS

(71) Applicant: The Procter & Gamble Company, Cincinnati, OH (US)

(72) Inventor: Samantha Chen-Yee WANG, Cincinnati, OH (US)

(21) Appl. No.: 16/926,824

(22) Filed: Jul. 13, 2020

Related U.S. Application Data
(60) Provisional application No. 62/874,670, filed on Jul. 16, 2019.

(51) Int. Cl. A61M 31/00; A61K 35/644; A61K 35/06; A61K 36/889; A61K 36/63; A61K 36/36

(52) U.S. Cl. CPC A61M 31/00; A61K 35/644; A61K 35/06; A61M 2210/1475; A61K 36/63; A61K 36/36; A61K 36/889

(57) ABSTRACT

The present disclosure generally relates to an applicator for administering a composition for treating one or more of vaginal dryness, vaginal irritation, vaginal itch, vaginal infection, vaginal odor, vaginal chafing, dyspareunia, and/or vaginal atrophy, and more specifically, to an applicator comprising a receptacle comprising a first portion, a second portion, and an attachment structure, where the first portion and second portion are removably attached to define a substantially spherical shape, where a vaginal care composition is stored within the receptacle.

### TECHNICAL FIELD

The present disclosure generally relates to an applicator for administering a composition for treating one or more of vaginal dryness, vaginal irritation, vaginal itch, vaginal infection, vaginal odor, vaginal chafing, dyspareunia, and/or vaginal atrophy, and more specifically, to an applicator comprising a receptacle comprising a first portion, a second portion, and an attachment structure, where the first portion and second portion are removably attached to define a substantially spherical shape, where a vaginal care composition is stored within the receptacle.

### BACKGROUND

Estimates indicate that by 2030 there will be about 1.2 billion menopausal and post-menopausal women in the world. Given that the average age at which menopause occurs has remained the same and that life expectancy among women has generally increased, the number of post-menopausal women is expected to grow. As such, there is increasing concern surrounding the conditions and symptoms experienced by peri-menopausal, menopausal, post-menopausal women, and the need for treatment therefore is growing as well.

As is well known, menopause is associated with a decrease in estrogen production. Decreased estrogen levels may result in changes to both the internal and external genitalia, including vaginal atrophy and a thinning of the vaginal and urethral mucous membrane, a loss in vaginal elasticity, and a reduction in gland secretion, which may be accompanied by a decrease in tissue hydration. Some symptoms of decreased estrogen levels may include vaginal infections, irritation, burning, dryness, itching, odor, chafing, atrophy, and pain during sexual intercourse (dyspareunia), thus, greatly impacting a woman's quality of life. For example, women may experience feelings of isolation, fear, resignation, anger, and a loss of libido and intimacy as a result. In addition to menopause, women may also experience a drop in estrogen levels or fluctuating hormones during breastfeeding, breast cancer hormonal treatment, and after surgical removal of the ovaries, pelvic radiation therapy for cancer, and chemotherapy.

There are a variety of solutions that have been proposed to address one or more of the above-described vaginal conditions and symptoms. Prescription-based remedies have included hormone replacement therapy, which can include an estrogen supplement with or without progesterone. In some instances, the hormonal therapies may be applied deep within the vaginal canal by plunger type applicators, for example, PREMARIN®, available from Pfizer, Inc., is supplied with a plunger type applicator for dispensing a cream into the vaginal canal; ESTRACE®, available from Allergan, Inc., also is supplied with a plunger type applicator; tablets may be similarly placed deep into the vaginal canal by a plunger type applicator, e.g., VAGIFEM®, available from Novo Nordisk Health Care AG, is supplied with an applicator to place a tablet within the vaginal canal, INTRAROSA™, available from Endoceutics, Inc., is supplied with a plunger type applicator to place inserts into the vaginal canal), or insertable rings (e.g., ESTRING® available from Pfizer, Inc., which is likewise inserted into the vaginal canal).

While hormonal therapies have shown positive effects, particularly in the treatment of vaginal atrophy, some women continue to experience the symptoms, and for many women, such treatment can prove to be too expensive. Hormone replacement therapy has also been reduced by contraindications such as a history of cancer and thromboembolism. Moreover, due to the nature of the condition, women may feel uncomfortable and/or embarrassed discussing the above-described symptoms and may avoid seeking a doctor's consultation. Additionally, many women stop annual visits to gynecologists, leaving their primary care family physician as the main resource, yet few primary care physicians address or treat menopausal symptoms since menopause is viewed as a “natural” process.

There are also several over-the-counter solutions that have been offered to consumers to treat various symptoms and/or conditions experienced by women. These include vaginal moisturizers (e.g., REPLENS® Long-Lasting Moisturizer and REPLENS® Moisture Restore External Comfort Gel, or HYALOGYN®/HYALOFEMME®, available from Fidia Farmaceutici SpA and both supplied with disposable applicators to place in the vaginal canal), lubricants for reducing discomfort during intimacy (e.g., REPLENS® Silky Smooth Personal Lubricant, ASTROGLIDE®, K-Y® gels and lubricants), wipes (e.g., VAGISIL® Anti-Itch Medicated Wipes), sprays, and washes and douches for eliminating bacteria that can cause unpleasant odors (e.g., SUMMER'S EVE®). The REPLENS® Long-Lasting Moisturizer, available from Church & Dwight Co., Inc., is provided with a plunger type applicator for depositing the moisturizer within the vaginal canal. The makers of REPLENS® have published a number of studies regarding the benefits of using the REPLENS® Long-Lasting Moisturizer (see, e.g., https://www.womenshealthcaresolutions.com/clinical-studies/replens/).

While the aforementioned over-the-counter solutions may be useful, some may not prioritize women's intimate health or the usage experience. Many of these solutions require a separate applicator and composition, which may add complexity and may be messy to use. Some options may have hygienic drawbacks, for example, not providing an applicator and requiring the user to apply the composition directly with her hands or providing an applicator but requiring frequent cleaning of the applicator, which may cause cross contamination of the vaginal care composition with cleaning and/or bodily fluids. Many over-the-counter plunger type applicators are designed to deliver a composition deep into the vaginal canal; such applicators typically deliver large doses of composition, which may subsequently leak out of the vaginal canal. Some over-the-counter products require a user to hold an applicator in an exact orientation, in order to properly dispense the composition inside the applicator. And, some products apply composition only to the vaginal canal and not to the introitus or external vaginal tissues. These drawbacks can make for an unpleasant experience and inhibit regular adoption for consistent habit formation.

There are opportunities for improvement. For example, it would be advantageous to provide applicators and methods for treating the vagina, particularly the vaginal introitus and, optionally, one or more external vaginal tissues, where the applicators have a vaginal care composition stored therein. It would also be advantageous to provide applicators that enable topical spreading of a composition onto the introitus and/or external vaginal tissues. It would be beneficial to provide an applicator that reduces the need for a female user to touch the composition or spread it about the applicator; to provide an applicator that can be held in any orientation without leaking, dripping, or otherwise improperly dispensing the composition; to provide an applicator having a geometry that enables topical spreading of the vaginal care composition without self-touch; and/or to provide a reusable, multi-use applicator.

### SUMMARY

The present disclosure relates to a product for treating the vagina, preferably the vaginal introitus and/or external vaginal tissues, comprising i) an applicator comprising a receptacle comprising a first portion, a second portion, and an attachment structure, where the first portion and second portion are removably attached to define a substantially spherical shape, and ii) a vaginal care composition stored within the receptacle, preferably where the applicator is hand-held.

The present disclosure also relates to methods of using the product described above, as well as kits comprising one or more of the products described above.

### BRIEF DESCRIPTION OF THE DRAWINGS

The above-mentioned and other features and advantages of the present disclosure, and the manner of attaining them, will become more apparent and the disclosure itself will be better understood by reference to the following description of non-limiting embodiments of the disclosure taken in conjunction with the accompanying drawings, wherein:

FIG. 1 is a perspective view of one example of a vaginal applicator of the present disclosure.

FIG. 2 is a perspective exploded view of the vaginal applicator of FIG. 1.

FIG. 3 is a schematic view of a portion of a receptacle of the vaginal applicator of FIG. 1.

FIG. 4 is a schematic representation of a hot pour process of manufacturing a vaginal composition having an arcuate surface.

FIG. 5 is a perspective top view of one example of an applicator of the present disclosure;

FIG. 6 is a front view of the applicator of FIG. 5 resting upright on a surface;

FIG. 7 is a cross-sectional view of the applicator of FIG. 6, taken along line L thereof;

FIG. 8 is a schematic view, depicting the applicator of FIG. 5 in use and in contact with the vaginal introitus;

FIG. 9 is a side view of the applicator of FIG. 6, illustrating various dimensions;

FIG. 10 is a schematic illustration of one use of the applicator of FIG. 5;

FIG. 11 is a front view of another example of an applicator, illustrating a grippable portion comprising a texture in the form of a plurality of grooves (the rear view, not shown, being the same as the front view);

Other features and advantages of the invention will be apparent from the following detailed description and from the claims.

### DETAILED DESCRIPTION

Various non-limiting embodiments of the present disclosure will now be described to provide an overall understanding of the principles of the function, design and use of the applicators, compositions, kits and methods disclosed herein. One or more examples of these non-limiting embodiments are illustrated in the accompanying drawings. Those of ordinary skill in the art will understand that the embodiments and methods described herein and illustrated in the accompanying drawings are non-limiting example embodiments and that the scope of the present disclosure is defined solely by the claims. The features illustrated or described in connection with one non-limiting embodiment can be combined with the features of other non-limiting embodiments. Such modifications and variations are intended to be included within the scope of the present disclosure.

All percentages are by weight of the vaginal care composition, unless specifically stated otherwise. All ratios are weight ratios, unless specifically stated otherwise. All ranges are inclusive of narrower ranges and combinable. Delineated upper and lower range limits are interchangeable to create further ranges not explicitly delineated. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity.

The compositions of the disclosure can comprise, consist essentially of, or consist of, the components as well as optional ingredients described herein. As used herein, “consisting essentially of means that the applicator, composition or component may include additional ingredients or features, but only if the additional ingredients or features do not materially alter the basic and novel characteristics of the claimed applicators, compositions or methods. As used in the description and the appended claims, the singular forms “a,” “an,” and the are intended to include the plural forms as well, unless the context clearly indicates otherwise. Unless otherwise defined, all technical and scientific terms used herein have the same meaning as commonly understood by one of ordinary skill in the art to which this invention belongs.

“Attachment structure” means any structure that is configured to engage and removably couple a first portion to a second portion.

“Cross-linked silicone rubber” means an elastomeric solid material resulting from cross-linking (e.g., sometimes referred to as “curing”) a liquid silicone rubber. Cross-linking of a liquid silicone rubber can occur by any process or means known in the art, including, but not limited to, cross-linking by addition (e.g., platinum), cross-linking by condensation or cross-linking with radicals (e.g., peroxide). Cross-linking may be facilitated by a cross-linking agent or temperature.

“Estrogen agent” means any natural or synthetic estrogen hormone (e.g., estrone, estradiol and estriol), metabolites thereof, esters thereof, analogues thereof; phytoestrogens (e.g., isoflavones, coumestans, prenylflavonoids); estrogen precursors (e.g., dehydroepiandrosterone); and/or any compound which binds to an estrogen receptor or which otherwise exhibits at least mild or weak estrogen-like effects, including selective estrogen receptor modulators (“SERM”) such as, e.g., afimoxifene (4-hydroxytamoxifen), arzoxifene, bazedoxifene, clomifene, femarelle (DT56a), lasofoxifene, ormeloxifene, raloxifene, tamoxifen, toremifene, mifepristone (RU486), VA2914, ulipristal, Proellex, Asoprisnil, and CDB-4124.

“External vaginal tissue” means one or more of the external female genital organs that are visible and bounded longitudinally by the mons pubis and anus and bounded laterally by the genitocrural folds, including but not limited to the vulvar vestibule, labia majora, labia minora, external urogenital tract, urethral orifice, clitoris, and vulvar skin.

“Fingertip offset distance” means the distance between (1) the hyponychium of the fingertip closest to the tip of an applicator while grasping the applicator during use, and (2) the tip of such applicator.

“Fluid communication” means that a flowable composition is movable between one structure or feature and a second structure or feature, either continuously or intermittently. Two structures are considered to be in fluid communication even though there are intermediate structures (e.g., a valve, pump, etc.) disposed between the two structures.

“Grippable,” in the context of the entire applicator or a portion thereof, means the applicator or portion thereof has an outer shape and size which may be grasped by two or more fingers or fingertips of a human hand to manipulate the applicator in use.

“Integrally constructed” and “integrally formed” mean broadly a structure or feature formed from two or more materials, pieces and/or parts that are not easily disassembled. In some embodiments, the materials, pieces and/or parts may be bonded (e.g., chemical, thermal or adhesive) or mechanically fastened together (e.g., inter-molded parts, interlocking parts). Some non-limiting manufacturing processes useful for forming the foregoing include injection molding, casting, bonding and printing processes.

“Liquid silicone rubber” means a cross-linkable liquid comprising a siloxane or silicone polymer (e.g., polysiloxanes, polydimethylsiloxanes (PDMS) and combinations thereof).

“Medical grade” means a material that passes either i) one or more of the United States Pharmacopeia and National Formulary (USP-NF) Class IV, V, and Class VI designations, or ii) ISO 10993 testing for one or more of cytotoxicity (ISO 10993-5), 7-day implant (ISO 10993-6), skin irritation (ISO 10993-10), and skin sensitization (ISO 10993-10).

“Micro-texture” means a texture or surface finish having a surface texture, Sq, from about 3 μm to about 30 μm, or from about 3.3 μm to about 20 μm, or from about 3.5 μm to about 10 μm as measured by the Surface Texture Procedure below.

“Peri-menopausal” woman is one who, in the absence of hormone replacement therapy or other medication, would experience a change in her intermenstrual cycle interval and have associated symptoms of estrogen deficiency, such as vasomotor flushes, vaginal dryness, and/or worsening premenstrual syndrome. Also, included are women who in the absence of hormone replacement therapy or other medication would experience less than 12 months of amenorrhea.

“Pharmacologically effective amount”, “therapeutically effective amount”, or simply “effective amount” means the amount of a composition, or ingredient thereof, effective to produce the intended pharmacological, therapeutic, or preventive result.

“Post-menopausal” woman is one who in the absence of hormone replacement therapy or other medication would experience at least 12 months of amenorrhea.

“Progesterone agent” means any natural or synthetic progesterone hormone, metabolites thereof, analogues thereof, progesterone precursors, and/or any compound which binds to a progesterone receptor or which otherwise exhibits at least mild or weak progesterone-like effects, including selective progesterone receptor modulators (“SPRM”) such as, for example, telapristone.

“Reservoir” means the space or volume which contains a vaginal care composition.

“Rotational symmetry” means the applicator, or a portion thereof such as the insertion portion or the grippable portion or the body, has an overall shape that looks the same (except with respect to its surface features) through some rotation (e.g., 45°, 90°, 135°, 180°, 225°, 270°, 315°, or 360°) about its longitudinal axis. For example, an applicator that has an overall shape that looks the same through a rotation of 45° is considered rotationally symmetrical through that 45° rotation. Likewise, an applicator that has an overall shape that looks the same through one full rotation is considered rotationally symmetrical through 360°. The reference to rotational symmetry herein, unless stated otherwise, ignores surface features such as print, coloring, coatings, text, graphics, dosing indicators, insertion indicators, surface textures, and surface finishes.

“Single-use applicator” means an applicator to be used once and disposed thereafter.

“Smooth” means a surface having a surface texture (Sq) less than about 30 μm, or less than about 10 μm, or less than about 3 μm as measured by the Surface Texture Procedure below. Smooth surfaces include surfaces having a micro-texture.

“Substantially free” means a component or material is present in amount less than 0.1%, 0.05%, 0.025%, 0.01%, or 0.001% by weight of the vaginal care composition.

“Taper” means to become smaller toward one end. An applicator, or feature thereof such as a first or second portion, may have a taper that is gradual, substantial, intermittent, continuous and combinations thereof. For example, an applicator is considered to taper from the maximum width to the tip merely if the bulk cross-sectional area (e.g., inclusive of both solid cross-sectional area and void cross-sectional area) at the tip is less than the bulk cross-sectional area at the maximum width, even though, for example, the bulk cross-sectional area may intermittently increase or remain constant between the maximum width and the tip.

“Vaginal care composition” means any composition that is suitable for application to the vaginal introitus and/or one or more external vaginal tissues.

“Vaginal introitus” or “introitus” means the opening of the vaginal canal adjacent to the vulvar vestibule.

### A. Applicator

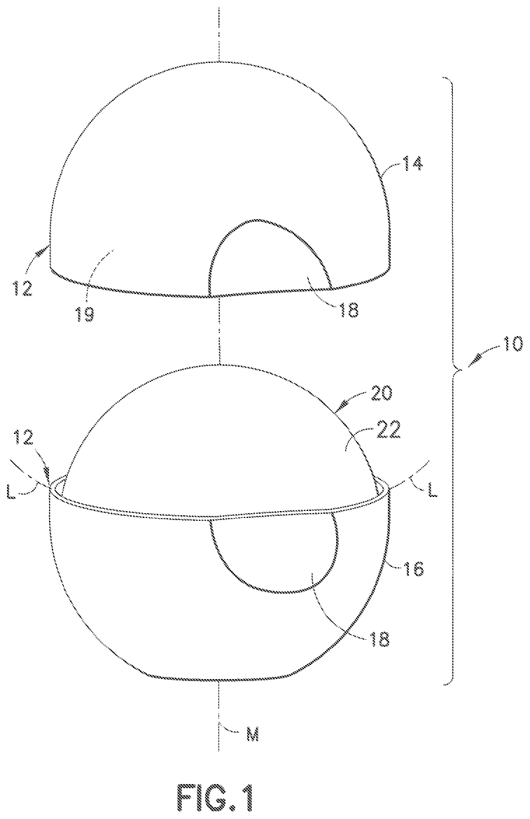

As is shown in FIG. 1, a non-limiting example of an applicator 10 containing a vaginal care composition 20 is described. As shown in FIG. 1, the applicator 10 comprises a receptacle 12 having the vaginal care composition disposed therein. The receptacle may be substantially spherical. Alternatively, the receptacle 12 may be substantially cylindrical with rounded surfaces, ovoid, ellipsoid, polyhedral, or the like. The receptacle 12 comprises a first portion 14 and a second portion 16, the portions being separable from each other along a line L that extends substantially circumferentially about the receptacle perpendicular to a major axis M extending longitudinally through the receptacle. The first portion 14 and/or second portion 16 may taper from line L. An interior surface 19 of the first portion 14 of the receptacle 12 is defined by a concave surface having a spherical or arcuate topography. Although various surfaces described herein are referred to as being spherical, it should be understood that the term “spherical” includes arcuate surfaces.

The first portion 14 may be removably attached to the second portion 16 via an attachment structure. Different attachment structures may be used to attach the first portion 14 to the second portion 16. The attachment structure may be disposed on the first portion 14, on the second portion 16, or on both. At least one of the first portion 14 or the second portion 16 may comprise either a recess or a protrusion that engage one another to removably attach the first portion 14 to the second portion 16. The recess and protrusion may be provided in the form of male and female threads, a bayonet fitment, or a groove and ring. Alternatively, the first portion 14 and the second portion 16 may removably engage by means of a friction or interference fit or magnets or other means, such as Velcro. For example, the first portion 14 may be removably attached by way of a friction fit or snap fit. The first portion 14 may be removably attached using a female snap-fit connection of the first portion 14, which engages a male snap-fit of the second portion 16 (not shown). The vaginal care composition 20 is stored in the receptacle 12. The applicator 10 may provide a better user experience by eliminating the need for a separate dispenser apart from the applicator (e.g., a hand pump).

The outer surface of the receptacle 12 may include a depression 18 formed partly on the first portion 14 and partly on the second portion 16. The depression 18 may be a divot formed in the receptacle 12 that accommodates the thumb of a user to facilitate the opening and closing of the receptacle by alternatingly detaching and attaching the first portion 14 and the second portion 16.

Upon separation of the first portion 14 from the second portion 16, the vaginal care composition 20 is revealed. The vaginal care composition 20 is mounted in the second portion 16 on a support platform and is defined by a quantity of vaginal care composition formed or otherwise configured to have a substantially spherical shape that includes a substantially spherical surface 22. The substantially spherical shape and substantially spherical surface 22 allow the vaginal care composition 20 to extend away from line L along major axis M toward a the tip 124 of the receptacle (as shown in FIG. 5), thereby enabling the application of the vaginal care composition to a user's vaginal introitus and/or one or more external vaginal tissues. The present disclosure is not limited to the vaginal care composition 20 being configured to have a spherical shape and spherical surface 22. The vaginal care composition 20 may be shaped and configured for application to the vaginal introitus and/or one or more external vaginal tissues. The vaginal care composition 20 may provide a smooth surface, and may be shaped, sized and oriented to allow for comfortable use.

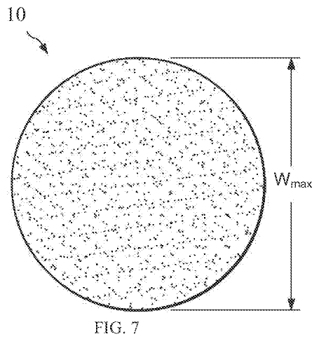

The applicator 10 may have a substantially circular cross-section (shown by way of example in FIG. 7) at line L, although other cross-sectional shapes at line L (e.g., ellipse, polygon, oval) are possible. The applicator 10 may be rotationally symmetrical about the major axis M, as this symmetry may facilitate easier usage and application of a vaginal care composition by a female user. The applicator 10 may have an overall shape that looks the same after an angular rotation thru a partial turn of 45°, 90°, 135°, 180°, 225°, 270°, 315°, or 360° about the major axis M.

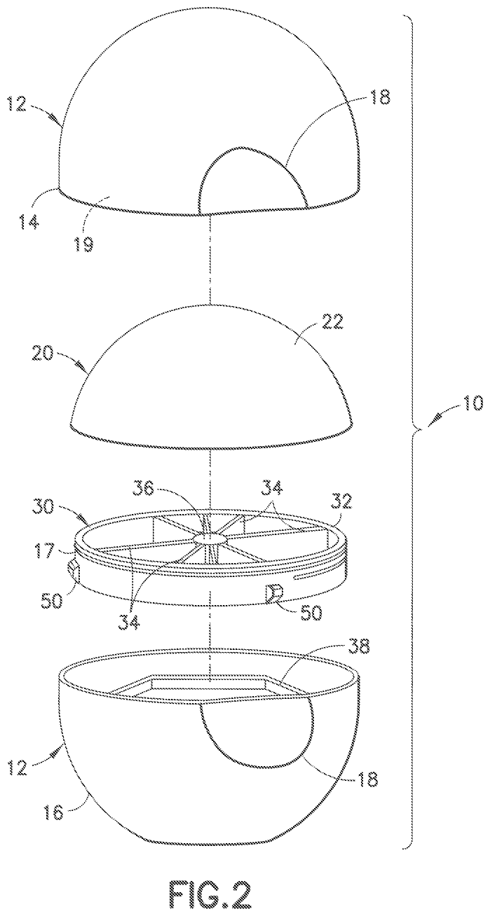

Referring now to FIG. 2, the vaginal care composition 20 may be mounted on a support platform 30. The support platform 30 may be a wheel having an outer rim 32 and threads 17 located thereon. A plurality of prongs 50 may be located along a lower edge of the outer rim 32 (the edge opposite the threads 17) to facilitate the connection of the support platform 30 to an interior connecting structure, such as ridges 38, in the interior of second portion 16. In the assembly of the receptacle 12, the prongs 50 may allow the support platform 30 to be snapplingly received on second portion 16.

The support platform 30 may include a plurality of spokes 34 that extend from the outer rim 32 and terminate at a hub 36. Both the widths of the spokes 34 and the diameter of the hub 36 are selected to provide a suitable area on which the vaginal care composition 20 can be supported. The spokes 34 may extend from the hub 36 at equal angles and connect with an inner surface of the outer rim 32. The disclosure is not limited to the use of spokes and hubs. The area bounded by the outer rim 32 may be solid. Although the outer rim 32 is described as being substantially circular, a portion along the edge of the outer rim may be flattened to allow for the accommodation of the depression 18.

One or more portions of the applicator may be formed in whole or in part from a thermoplastic elastomer, a natural rubber, metal (e.g., aluminum, stainless steel), a synthetic rubber (e.g., a silicone elastomer/silicone rubber), a polyester (e.g., a polyurethane, such as STERALLOY™ 2021-5 available from Hapco, Inc.) and/or a thermoplastic. The material used to form the applicator is preferably medical grade. Some examples of suitable thermoplastics include polypropylene, high density polyethylene, low density polyethylene, polystyrene, polyethylene terephthalate, acrylonitrile butadiene styrene, nylon, polycarbonate, and polyacrylic. Alternatively, the one or more portions of the applicator may be formed from an open cell foam.

Portions of the applicator (e.g., the first portion and/or the second portion) or the applicator overall may also have a low absorbency so that the applicator may be easily cleaned and remain sanitary due to little or no absorption of the vaginal care composition, cleaning fluids and/or bodily fluids by the applicator. In some examples, the applicator (or portions thereof) exhibits an absorbency less than 10% or from about 0.01% to about 2%, or from about 0.05% to about 1%.

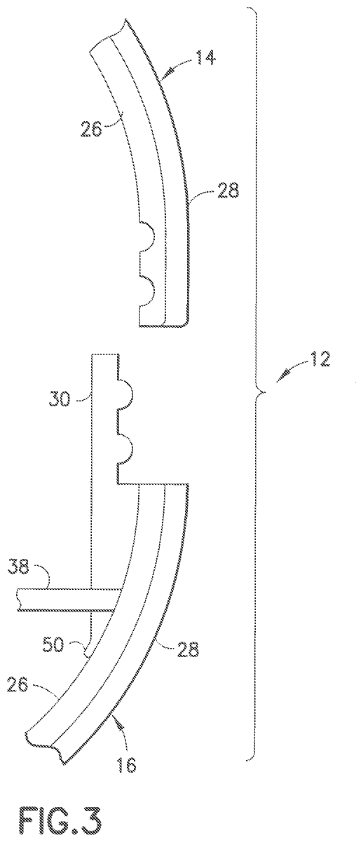

Referring now to FIG. 3, both the first portion 14 and the second portion 16 of the receptacle 12 may comprise a thermoplastic elastomer (TPE) injection molded over a plastic form comprising acrylonitrile-butadiene-styrene (ABS) copolymer. Accordingly, the defining surfaces of both the upper portion 14 and the lower portion 16 may comprise a first layer 26 of ABS copolymer covered by a second layer 28 of TPE. The disclosure is not limited to the first layer 26 being ABS copolymer, however, as the first layer in one or both of the first portion 14 and the second portion 16 may be polyethylene (PE), providing a different tactile sensation to the user upon handling the receptacle 12. In any configuration, the material of the second layer 28 may provide a desirable tactile sensation to the user of the applicator 10, namely by providing a smooth, grippable surface, thereby facilitating the easy opening and closing of the receptacle 12.

The spherical outer surface 22 of the vaginal care composition 20 comprises a mound-shaped quantity of the vaginal care composition. The spherical or mound shape provides desirable tactile sensations and use benefits as compared to other forms of vaginal care composition (e.g., vaginal care composition may be applied to the vaginal introitus and/or one or more of the vulva, vestibule, labia majora, labia minora, and/or external urogenital tract while limiting the vaginal care composition from contacting the user's fingers during use). Desirable tactile sensations result from the spherical shape having a larger exposed surface area (as compared to the other forms of the vaginal care composition) and the tactile sensation of a curved surface against the vaginal tissues. The outer surface 22 may be smooth, so as to avoid irritation of the vaginal tissues during manipulation of the applicator to administer the vaginal care composition to the vaginal tissues of interest.

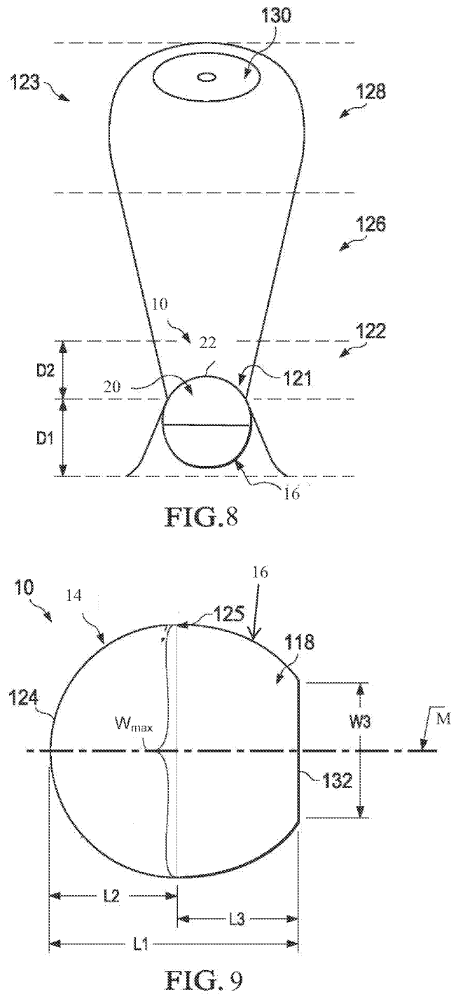

The applicator 10 and particularly the outer surface 22 of the vaginal care composition may be shaped and sized to accommodate the anatomical geometry of the vaginal introitus and for applying the vaginal care composition thereto. While there may be considerable variability in vaginal shape, axis, and dimension from woman to woman, the dimensions and structural attributes of the applicator 10 and the outer surface 22 may be designed to accommodate the vaginal introitus, taking into consideration a wide range of anatomical measurements while also self-limiting the insertion depth of the applicator. Since the applicators may be used by women suffering from vaginal atrophy, the size and shape should also accommodate the anatomical changes that occur with vaginal atrophy. In a study by Luo et al. entitled, “Quantitative analyses of variability in normal vaginal shape and dimension on MR images” (“Luo et al.”), magnetic resonance imaging (MRI) was used to take a series of measurements in order to quantify variability in vaginal dimensions for a group of women age 28 to 70. Luo et al. recognized that the vagina has three regions: a lower region (distal half of an anterior vaginal wall (AVW)), a middle region (proximal half of the AVW), and an upper region (cervical portion axis). Among its measurements, Luo et al. also assessed vaginal widths along a vaginal length. Luo et al. notes that the vaginal width is generally at its largest in an upper region of the vagina but generally decreases toward a lower region, such that the width is at its narrowest at the vaginal introitus. Lou et al. noted that the vaginal threshold (introitus) had a minimum dimension of 9 mm, maximum of 31 mm and mean of 17 mm, with a standard deviation of 5 mm, which may represent a dimensional range for peri- and post-menopausal woman. The schematic view depicted in FIG. 8 illustrates a narrowing vaginal width along a vaginal length to the vaginal introitus 121, which is shown to be in contact with the applicator 10. The outer surface 22 is shown in FIG. 8 to extend a minimal distance thru the vaginal introitus.

The applicator 10 may store from about 1 g, 2 g, 5 g, or 10 g to about 5 g, 10 g, 20 g, 50 g, 100 g, or 200 g of vaginal care composition. The applicator 10 may hold a sufficient quantity for multiple doses/uses over several days or weeks. The applicator may store from about 1 g to 200 g, or from about 5 g to about 100 g or from about 10 g to about 50 g. The dosage of the vaginal care composition dispensed onto vaginal tissue may be from about 0.1 g to about 2 g, from about 0.2 g to about 1.2 g, or from about 0.3 g to about 1.1 g per usage of the applicator. The applicator may be refilled between uses, but preferably is not refilled between uses. In some examples, the dosage is about 0.5%, or about 1%, or about 2%, or about 3%, or about 5% to about 50%, or about 25%, or about 15%, or about 10% by weight of the vaginal care composition originally stored in the applicator.

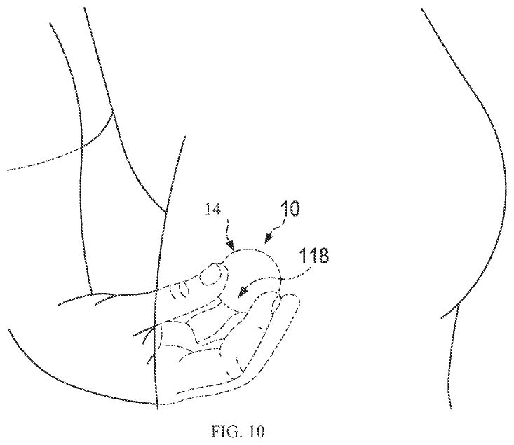

Referring to FIG. 9, the first portion 14 may have a length L2, where the length L2 is intended to correspond to the desired depth of insertion of the vaginal care composition 20 into the vaginal introitus 121 during use. The receptacle 12 has a maximum width Wₘₐₓ, which is intended to correspond to the inner dimensional width of the vaginal introitus opening 121, taking into account the various dimensions of the vaginal introitus 121 across a population of users, including women suffering from vaginal atrophy. With respect to an applicator 10, the length L2 of the first portion 14 refers to the longitudinal distance extending from the tip 124 of the receptacle toward the location of the base 125 of the first portion, shown FIG. 9. The terminal end of the length L2 opposite the tip 124 defines the location of the base 125 of the first portion 14. Stated differently, the base 125 of the first portion 14 may be located a distance L2 from the tip 124 of the receptacle 12. The length L2 of the first portion 14, taken along the major axis M, may be from about 10 mm to about 40 mm, or from about 10 mm to about 35 mm, or from about 10 mm to about 30 mm, or from about 10 mm to about 25, from about 10 mm to about 20 mm, where such lengths may correspond to an estimated distance from the vaginal introitus 121 to a proximal portion of the lower region 122 (e.g., D2 of FIG. 8). The maximum width Wₘₐₓ of the receptacle 12 is the width taken at the location of the base 125 (which is defined by the terminal end of the length L2 as previously described).

The length L3 of the second portion 16, taken along the major axis M, may be from about 10 mm to about 40 mm, or from about 10 mm to about 35 mm, or from about 10 mm to about 30 mm, or from about 10 mm to about 25, from about 10 mm to about 20 mm. The maximum width Wₘₐₓ can be from about 20 mm to about 80 mm, or from about 20 mm to about 60 mm, or from about 25 mm to about 70 mm, or from about 25 mm to about 55 mm, or from about 30 mm to about 50 mm or any value from about 20 mm to about 80 mm, or any range formed by any of the preceding values. The Wₘₐₓ may be selected to limit the distance the applicator 10 is inserted into the vaginal introitus using gentle hand pressure (and without experiencing discomfort) and/or to signal to a user that the appropriate insertion depth has been achieved during use. It may be desirable for the applicator to be dimensioned to prevent over-insertion of the applicator into the vaginal canal. For example, the first portion 14 or the second portion 16 of the receptacle 12 may further exhibit dimensions that provide a self-limiting feature with respect to insertion beyond the lower region 122 of the vagina 123, such that in some examples, contact between the first portion 14 and either of a middle region 126 or an upper region 128, including the cervix 130, is avoided, as illustrated in FIG. 8.

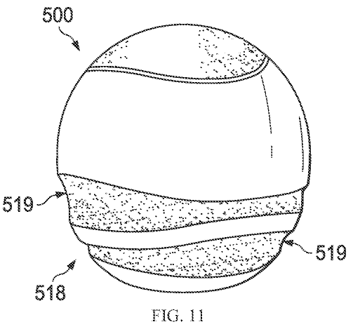

Referring again to FIG. 9, a grippable portion 118 may be provided adjacent the bottom 132 of the applicator 10. In some examples, the grippable portion 118 may extend the length L3 of the second portion 16 (FIG. 9) from the bottom 132 to the location of the base 125. The bottom 132 has a width W3, which may be, in some examples, a width sufficient to allow the applicator 10 to remain standing in an upright position on a surface (e.g., a countertop), as shown, for example, in FIG. 2, without falling over. The width W3 is about 80 mm or less, or from about 15 mm to about 60 mm or from about 20 mm to about 40 mm. A bottom 132 having a width within these ranges may be desirable as these values provide a starting size for the grippable portion 118 that is comfortable for gripping with at least 2 fingertips. In some instances, the grippable portion may be grasped by 3, 4 or 5 fingertips. Thus, while holding the applicator 10 at the grippable portion 118, a female user may apply the vaginal care composition to the desired vaginal tissue by contacting at least the vaginal introitus and, optionally, one or more of the vulva, vestibule, labia majora, labia minora, and/or external urogenital tract with the first portion 14 such that at least a portion of the vaginal care composition is transferred to the vaginal tissue. One non-limiting illustration of the manner of grasping a grippable portion 118 and inserting the applicator 10 into the vaginal introitus is shown in FIG. 10, where the applicator is illustrated as grasped by 5 fingertips. In some instances, the applicator is grasped by only the fingertips (versus the palm of the hand or lower finger knuckles) to permit easy manipulation of the applicator and administration of the vaginal care composition to the vaginal tissues of interest.

As described herein, the grippable portion 118 preferably remains external to the labia during use, which assists with keeping the fingers of the female user clean during use of the applicator. In some examples, the longitudinal distance L3 from the bottom 132 to the location of the base 125 may be from about 5 mm to about 65 mm, or from about 10 mm to about 65 mm, or from about 10 mm to about 50 mm, or from about 20 mm to about 40 mm, or any value from about 10 mm to about 65 mm, or any range formed by any of the preceding values. The grippable portion may be defined by a texture or visual indicator, a grippable portion configuration having a texture is shown in FIG. 11. The texture may comprise a plurality of protrusions, a plurality of recesses and combinations thereof. The texture may be provided as a plurality of ridges, a plurality of grooves and combinations of the foregoing. The grippable portion may alternatively or in addition thereto be provided with a surface finish (e.g., micro-texture) or surface treatment (e.g., dip coating, spray coating, printing) that enhances the grippability of the grippable portion. The texture, surface finish or surface treatment may partially or wholly encircle the body. The texture may have a maximum height or maximum depth from about 0.1 mm to about 5 mm, or from about 0.1 mm to about 4 mm, or from about 0.25 mm to about 3 mm, or from about 1 mm to about 2 mm or any value from about 0.1 mm to about 5 mm or any range formed by any of the preceding values. The texture may be provided as a repeating, preferably regularly repeating, pattern, although random or irregular patterns may also be provided. Further, the texture may be provide in the shape of objects, some non-limiting examples being leaves, flowers, stars and geometric shapes. The texture may also be provided as a repeating, closed geometric shape, some non-limiting examples being circles, diamonds, triangles, rectangles and combinations thereof.

One non-limiting example of a texture is depicted in the applicator 500 of FIG. 11, where the grippable portion 518 comprises a plurality of grooves 519 arranged in a repeating pattern. While FIG. 11 illustrates a texture that extends about the entire circumference or lower outer surface of the applicator, the disclosure is not so limited. The texture or surface finish/surface treatment may extend about only a portion of the circumference or lower outer surface of the applicator.

The texture, surface finish or surface treatment may extend about 10 mm or more from the bottom 132 of the applicator. These features may identify the grippable portion to a user and therefore also function as a visible indicator of the location of the grippable portion. In some examples where grooves or ridges are provided, a groove or ridge width may be greater than the groove or ridge height to minimize gathering or collection of the vaginal care composition and/or provide for easy rinsing and cleaning of the applicator after use.

The bottom 132, or a portion thereof, may be substantially flat. As shown for example in FIG. 9, the bottom 132 of applicator 10 may include a flat portion that can allow a user to stand the applicator 10 in an upright position on a surface before and after use, such that it can be oriented in a way to minimize or prevent contact between a resting surface and the vaginal care composition 20. The bottom of the applicator 10 may comprise an indentation (not shown) which is partially or wholly encircled by a flat portion on the bottom 132, which can allow a user to further minimize or prevent contact between a resting surface and the applicator 10. While the bottom 132 of the applicator 100 is shown in FIG. 9, for example, as having a substantially flat or planar portion, the disclosure is not so limited and the bottom 132 may be provided in other shapes, such as, for example, rounded, partially rounded or any other suitable shape. Likewise, while the grippable portions illustrated, for example in FIG. 11, is shown as tapering from the maximum width Wₘₐₓ to the bottom 132, the shape of outer surface of the grippable portion may be provided in other shapes (e.g., non-tapered).

Referring to FIG. 9, the overall length L1 of the applicator 10 may minimize or prevent contact between a female user's fingers and the vaginal areas described above while providing for an appropriate insertion depth, e.g., slightly beyond the vaginal introitus. In some examples, the length L1 of the applicator may be from about 20 mm to about 90 mm, or from about 35 mm to about 85 mm, or from about 30 mm to about 80 mm, or from about 40 mm to about 70 mm, or from about 45 mm to about 65 mm or any value from about 20 mm to about 90 mm or any range formed by any of the preceding values. The ratio of the length L1 of the applicator to the maximum width Wₘₐₓ of the applicator may be from about 1:2 to about 1:1 or any range formed by any of the preceding values. It is believed that the aforementioned lengths, widths and ratios may provide an applicator that balances an appropriate insertion depth, provides a grippable portion that may remain external to the vaginal tissues of interest, reduces the risk over-insertion (including “losing” the applicator within the vaginal canal during use), and facilitates appropriate spreading of the vaginal care composition about the vaginal tissues of interest, while taking into account anatomical variability between female users, including those experiencing vaginal atrophy.

The applicators may be devoid of moving parts, electrically powered elements (e.g., motors, lights, circuits, buzzers, speakers, etc.) and/or electrical power sources (e.g., batteries) to provide an applicator that is simple to manufacture and also easily immersed in or rinsed with water for cleaning. An applicator that is devoid or substantially free of batteries and/or electrically powered elements (e.g., motors, lights, etc.) is referred to as a non-electrical applicator. The applicator may be not phallic shaped so its appearance is more like a beauty care inspired device rather than a sexual or medicinal device, which may be less intimidating and can inspire long term habit adoption by a female user.

Applicators that include a vaginal care composition within the applicator are preferred, because such applicators eliminate the need for a user to dispense the vaginal care composition from a separate dispenser apart from the applicator (e.g., a hand pump). However, solid applicators that are substantially spherical in shape may also be used. Such a solid applicator may be used in combination with a separate dispenser containing a vaginal care composition. In use, a female user may grasp the applicator, depositing an amount of the vaginal care composition from the dispenser on at least a portion of the applicator, manipulating the applicator to administer the vaginal care composition to her vaginal introitus and, optional, external vaginal tissues such as at least one of the vulva, vestibule, labia majora, labia minora, external urogenital tract, such that at least a portion of the vaginal care composition is transferred to the vaginal tissues of interest.

In such solid applicators at least 60%, 70%, 80%, 90%, 95% or 100% of the bulk volume may be solid volume, preferably formed from a homogenous solid. Further, the applicator may be devoid or substantially free of chambers for storing or containing a composition and/or passages, conduits, channels and/or openings or ports for delivering such a composition. Similarly, the solid applicator may be devoid of plungers and other structures that may be used to expel a vaginal care composition from the body of the applicator.

The solid applicator may be combined with a vaginal care composition in a kit. The kit may comprise a single applicator that is reusable, or a plurality of applicators may be provided. The kit may further include a dispenser, in which the vaginal care composition is stored. The dispenser is separate from the applicator. The kit may further include a pouch, a cap, or a case, in which the applicator and/or dispenser may be stored before and after use. The applicator, dispenser containing the vaginal care composition and the pouch may be packaged together and sold as a packaged unit or sold separately.

Such solid applicators differ in shape from the applicators described in Application Serial No. US2019/015149, which is hereby incorporated by reference, but are otherwise similar to the applicators described in Application Serial No. US2019/015149.

### B. Vaginal Care Compositions

The applicator 10 may store a wide variety of vaginal care compositions suitable for application to the vaginal introitus and, optionally, external vaginal tissues for treating vaginal dryness, vaginal irritation, vaginal itch, vaginal infection, vaginal odor, vaginal chafing, dyspareunia, and/or vaginal atrophy. In some examples, the vaginal care compositions are preferably non-irritating and substantially free of ingredients that are less suitable for application to these tissues. The vaginal care composition may be provided as a solid or a semisolid, a state somewhere between a solid and a liquid. In some examples, the vaginal care composition provides a moisturization and/or hydration benefit to one or more vaginal tissues. In some examples, the vaginal care composition may comprise an estrogen agent and/or a progesterone agent.

The applicators and methods described herein may be particularly useful for treating or ameliorating vaginal dryness, vaginal irritation, vaginal itch, vaginal infection, vaginal odor, vaginal chafing, vaginal atrophy, and/or other conditions, for which treatment over an extended period of time (e.g., daily use over multiple days or weeks) is desirable. Without wishing to be bound by theory, it is believed that vaginal dryness and/or atrophy may be best treated by applying a vaginal care composition suitable for treating or ameliorating vaginal dryness and/or atrophy to the user's vaginal introitus and, optionally, one or more external vaginal tissues over an extended period of time (e.g., daily use over multiple days or weeks). Applicators capable of storing one or more doses of vaginal care composition may increase consumer compliance over extended periods of time by making the process more convenient and intuitive.

The vaginal care composition may be provided in the form of an oil-in-water emulsion with one or more optional additional ingredients. An oil-in-water emulsion may provide a sensorial feel that is light and non-greasy, but still delivers moisturization and lubricity without aesthetic negatives like stickiness or heavy residue.

The vaginal care composition for treating vaginal dryness and/or vaginal atrophy may comprise water, one or more oils, and, optionally, one or more vitamins and/or pro-vitamins (e.g., a substance that may be converted to a vitamin by metabolic processes), one or more thickeners, one or more emulsifiers, one or more humectants, one or more lubricants (which may also be in the form of an oil), one or more moisturizers (which may also be in the form of an oil), one or more feel modifiers (e.g., particulates, powders, and film forming agents), one or more preservatives, and one or more pH adjusting agents. The vaginal care composition preferably provides a suitable viscosity, dry feel, moisturization/emolliency benefits, suitable lubricity and/or vaginal skin health benefits.

The vaginal care composition may comprise one or more waxes, such as beeswax. The vaginal care composition may comprise from about 1%, or from about 2%, or from about 5%, or from about 10% to about 99%, or to about 90%, or to about 80%, or to about 70%, or to about 60%, or to about 50%, or to about 40%, or to about 30%, or to about 20% by weight of the vaginal care composition of wax.

The vaginal care composition may comprise one or more oils, including botanical oils, silicone oils and other oils. The vaginal care composition may comprise from about 1%, or from about 2%, or from about 5%, or from about 10% to about 99%, or to about 90%, or to about 80%, or to about 70%, or to about 60%, or to about 50%, or to about 40%, or to about 30%, or to about 20% by weight of the vaginal care composition of oil.

Botanical oils may be derived from one or more plant source materials, such as leaf, root, bark, stem, flower or seed of a plant. The botanical oil may be a seed oil, a nut oil, a flower oil, or a leaf oil. The botanical oil may comprise polyunsaturated fatty acids, preferably omega-3 (e.g., α-linolenic acid) and/or omega-6 fatty acids. The botanical oil may be a coconut oil, a sunflower seed oil, a safflower oil, and combinations thereof, which may be rich in omega-3 and/or omega-6 fatty acids. The botanical oil may provide an emolliency vaginal skin care benefit. The vaginal care composition may comprise from about 1%, or from about 2%, or from about 5%, or from about 10% to about 99%, or to about 90%, or to about 80%, or to about 70%, or to about 60%, or to about 50%, or to about 40%, or to about 30%, or to about 20% by weight of the vaginal care composition of botanical oil(s).

The vaginal care composition may further comprise one or more silicone oils. Silicone oils are liquids at ambient conditions comprising one or more polymerized siloxanes or silicone polymers (e.g., polysiloxanes, polydimethylsiloxanes (PDMS), and combinations thereof). The silicone oil may comprise dimethicone, dimethiconol (a high molecular weight silicone gum), cyclomethicone, cyclopentasiloxane, and combinations thereof, one example is Dow Corning® 1503 Fluid which comprises a combination of dimethicone and dimethiconol. The vaginal care composition may comprise from about 0.1% to about 40%, or from about 0.5% to about 35%, or from about 1% to 30% by weight of the vaginal care composition of silicone oil(s).

The vaginal care composition may comprise one or more vitamins and/or pro-vitamins for providing a vaginal skin health benefit. Some non-limiting examples include niacinamide, panthenol, vitamin B3, vitamin B5, vitamin E, and derivatives thereof. The vaginal care composition may comprise from about 0.1% to about 7%, or from about 0.5% to about 5%, or from about 2% to about 4% by weight of the vaginal care composition of vitamin(s) and/or provitamin(s).

The vaginal care composition may comprise one or more humectants, such as glycerol. The humectants may also provide a moisturizing benefit. The humectant may be provided in an amount from about 0.01% to about 20%, or from about 0.1% to about 15%, or from about 1% to about 12% by weight of the vaginal care composition. Other humectants which may be provided include other polyhydroxy alcohols (e.g., sorbitol, propylene glycols, butylene glycols, pentylene hexylene glycols), polyethylene glycols, aloe vera, in any of its forms, hyaluronic acid, and combinations thereof. The vaginal care composition may further comprise one or more solid micro-particles, such as silicone particulates/silicone powders, to impart desirable feel characteristics to the vaginal care composition. The vaginal care composition may comprise siloxane particles (e.g., polymethylsilsesquioxane) having an average particle size from about 1 μm to about 15 μm. The particles may be in the form of mono-disperse microspheres. The polymethylsilsesquixane particles are sometimes also referred to as silicone resins.

The vaginal care composition may further comprise one or more emulsifiers, and/or one or more thickeners. Some non-limiting examples of emulsifiers include cationic surfactants, anionic surfactants, non-ionic surfactants, polyethylene glycol, polyethylene glycol polypropylene copolymers, alkyl glucosides, and fatty alcohols. Some non-limiting examples of thickeners include gums, starches, modified starches, clays, cross-linked water swellable polymers, cetearyl glucoside, cetearyl alcohol, behenyl alcohol, cetyl alcohol, stearyl alcohol, polyacrylates, and polyacrylamide. The thickeners are provided in amounts to facilitate achieving the desired viscosity in combination with the other ingredients. The thickeners may be provided in an amount from about 1% to about 10%, or from about 2% to about 8%, or from about 4% to about 8% by weight of the vaginal care composition.

The vaginal care composition may comprise petrolatum, beeswax, carnauba wax, coconut oil, olive oil, and/or jojoba oil or mixtures thereof, which can be eroded when drawn over a surface (e.g., external vaginal tissues). The vaginal care composition may comprise petrolatum and/or petrolatum-based compounds. The vaginal care composition may comprise petrolatum, beeswax, carnauba wax, coconut oil, olive oil, jojoba oil, and an ingredient selected from the group consisting of emollients, moisturizers, vitamins, aloe vera, colorants, fragrances, and mixtures thereof.

The vaginal care composition may have a viscosity conducive to spreading onto the vaginal tissues of interest using the applicators described herein without undue effort. The vaginal care composition may exhibit a viscosity of from about 2,000 cps to about 2,000,000 cps, or from about 5,000 cps to about 1,000,000 cps, or from about 20,000 cps to about 800,000 cps, or from about 40,000 cps to about 800,000 cps, or any value from about 2,000 cps to about 2,000,000 cps when measured at 25° C., or any range formed by any of the preceding values.

The vaginal care composition may have a viscosity conducive to filling, processing, and manufacturing applicators as described herein. The vaginal care composition may exhibit a viscosity of from 10 cps to about 200,000 cps, preferably from about 15 cps to about 100,000 cps, more preferably from about 20 cps to about 50,000 cps, even more preferably from about 25 cps to about 40,000 cps when measured at 70° C., or any range formed by any of the preceding values.

The vaginal care composition may be substantially free of retinol, retinyl esters, retinaldehyde, peptides, ethanol, sunscreens, and sensates. In some examples, the composition may be substantially free of perfumes and pigments. In some examples, the composition may be substantially free of particulates for exfoliation. In such examples, the excluded particulates have an average particle size of from about 125 microns to about 700 microns or more. Examples of such particulates may include polyethylene terephthalate (PET) microbeads, crushed apricot kernel shells, salt crystals, sugar crystals, and crushed volcanic rock. It is believed the foregoing ingredients may either be irritating or present an unsatisfactory user experience in a vaginal care composition. In some examples, the vaginal care composition may be substantially free of the combination of carbomer and polycarbophil and other bioadhesives or mucoadhesive ingredients.

### C. Method of Making

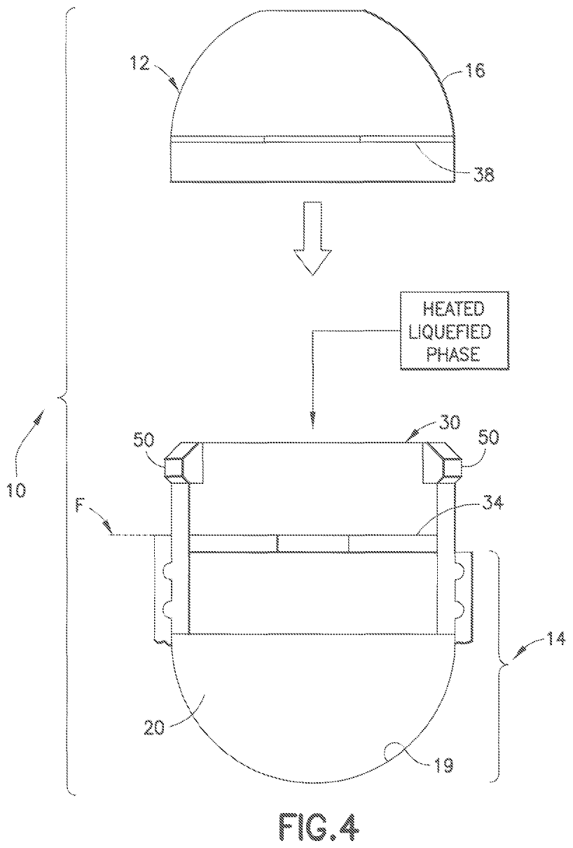

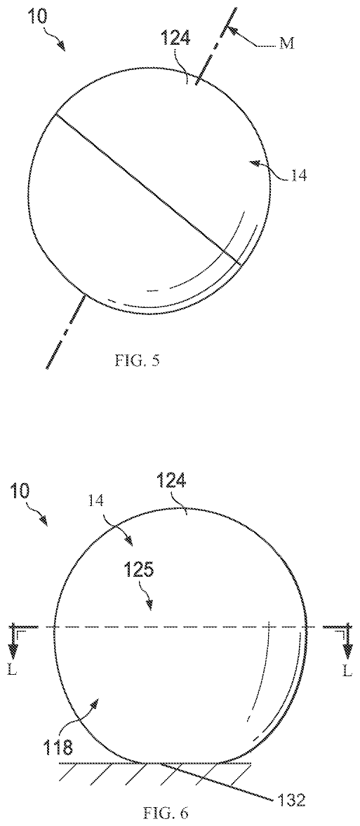

Referring now to FIG. 4, the vaginal care composition 20 may be made using a hot pour process. In using the hot pour process, the ingredients of the vaginal care composition are combined and heated in a liquefied phase and poured into the final consumer packaging, which in this case is the receptacle 12. In preparation for the hot pour process, the support platform 30 is assembled with the first portion 14 and inverted such that the support platform is level and vertical with regard to the first portion. Orienting the assembled first portion 14 and support platform 30 in this manner allows for the heated and liquefied phase to be poured into the first portion 14 and retained therein. The interior surface 19 of the first portion 14 may be coated with a release agent prior to pouring the heated and liquefied phase, which thereby allows for efficient and clean removal of the first portion by the user when using the finished product.

After the heated and liquefied phase is poured into the first portion 14 and filled to a level F to cover at least a portion of the spokes 34, the liquefied phase is cooled or allowed to cool. The spokes 34 provide surfaces onto which the vaginal care composition can adhere, as it solidifies. The disclosure is not limited to the solidification of the vaginal care composition around spokes, however, as the support platform 30 can include any suitable configuration of surfaces on which the vaginal care composition can solidify. Upon sufficient solidification of the vaginal care composition, the second portion 16 is inserted onto the assembled and filled first portion 14 and support platform 30.

The use of the hot pour method in manufacturing the vaginal care composition 20 defined by the spherical surface 22 provides cost benefits over similar methods that use the bullet method. In particular, using the hot pour method obviates the need for an intermediate step in which hot liquid is poured into separate molds, thereby also eliminating the need for additional equipment (molds). Furthermore, the additional steps of removing solidified product from molds and processing and handling the solidified product are avoided.

### D. Methods of Use

The applicators and vaginal care compositions may be used in a variety of ways. A method for treating vaginal dryness and/or vaginal atrophy may include a female user grasping the applicator, detaching the first portion 14, manipulating the applicator to administer the vaginal care composition to her vaginal introitus and, optional, external vaginal tissues such as at least one of the vulva, vestibule, labia majora, labia minora, external urogenital tract, with the applicator such that at least a portion of the vaginal care composition thereon is transferred to the vaginal tissues of interest.

The methods described herein may be directed to and/or performed by women experiencing vaginal dryness and/or vaginal atrophy and/or suffering from a reduction in estrogen levels. Vaginal dryness may also be due to, for example, vaginitis, inflammation of the vagina due to thinning and shrinking of the tissues, sexual arousal disorder, menopause, drug (prescription or over the counter) induced vaginal dryness, dyspareunia, sexual pain disorder, pregnancy, breast feeding, hormone imbalance, anxiety and diabetes. In addition, the applicators and vaginal care compositions may have non-medical uses for females in need of vaginal lubrication. Vaginal atrophy (sometimes referred to as atrophic vaginitis or vaginitis, vulvovaginal atrophy, or urogenital atrophy) may be characterized by thinning and shrinking of tissues as well as decreased lubrication, typically induced by a reduction in estrogen which happens naturally in perimenopausal, menopausal and post-menopausal women. Symptoms may include vaginal soreness, itching, dryness, and painful intercourse. In some examples, the female user is 40, 45, 50 or 55 years of age or older, experiencing a reduction in estrogen levels, suffering from vaginal atrophy, vaginal dryness, and/or is perimenopausal, menopausal or post-menopausal. It is believed that performance of the methods described herein using an applicator and vaginal care composition may provide the female user with an acute and/or a chronic reduction in one or more of vaginal dryness, pain during sexual intercourse, vaginal itching and vaginal irritation.

To treat female genital dryness and/or vaginal atrophy, a female user may grasp or hold the applicator and manipulate the applicator to administer the vaginal care composition to her vaginal introitus and, optionally, at least one of the vulva, vestibule, labia majora, labia minora, external urogenital tract, using the insertion portion such that at least a portion of the vaginal care composition thereon is transferred to the vaginal tissue. The female user may be seated, standing, laying down, squatting, or have one leg supported by a surface during manipulation of the applicator. The female user grasps the grippable portion of the applicator with 2, 3, 4, or 5 fingertips, preferably with the pads of the fingertips (one example being shown in FIG. 10). The female user may grasp the applicator using only her fingertips, and not her fingernails or the palm of her hand or lower knuckles in order to provide better control over manipulation of the applicator. In some examples, the grippable portion is deformable (e.g., the material used to form it has a Shore A hardness or the grippable portion has a peak compressive force) so that the female user may slightly compress the grippable portion, for example by 0.5 mm to about 2 mm, using her fingertip pads. This permits the female user to more easily grasp the applicator and ergonomically manipulate the applicator in a variety motions to administer the vaginal care composition to the vaginal introitus and, optionally, external vaginal tissues. The female user may grasp the applicator from any orientation and manipulate the applicator in a wide variety of manners without accounting for the orientation of the applicator within her hand. This provides easy and quick performance of the method of treatment. The female user may insert the applicator into her vaginal introitus a distance of about 25 mm or less to apply the vaginal care composition to her introitus.

The applicator is preferably only inserted far enough to treat the vaginal introitus without discomfort. The applicator is inserted only so far as is comfortable to avoid pain or tearing or bleeding from the delicate tissues. The female user may insert the applicator into the vaginal introitus a distance of 40 mm, 35 mm, 30 mm, 25 mm or less, or from about 30 mm to about 5 mm or from about 25 mm to about 10 mm. The fingertip offset distance when the female user grasps the applicator is from about 30 mm to 65 mm, or from about 30 mm to about 60 mm, or from about 30 mm to about 55 mm. Without wishing to be bound by theory, it is believed that consumers will be adverse to self-touch and soiling their fingers with bodily fluid and/or the vaginal care composition. As such, applicators designed to be gripped with the fingertip offset distances described above will result in the applicator being inserted minimally into the introitus during use.

The female user may administer the vaginal care composition to the vaginal tissues of interest for a period of time from about 1 second to about 40 seconds or about 1 second to about 30 seconds or from about 1 second to about 15 seconds or any range formed by any of the preceding values. A short administration time is desirable for providing a method that is convenient and quick to complete.

Since the experience of both the applicator and vaginal care composition may be useful for encouraging long term habit adoption by a female user for both acute and chronic treatment of vaginal dryness and/or vaginal atrophy, the female user may use the applicator to apply the vaginal care composition on a regular basis. With respect to frequency of administration, in some examples, the method may be performed by a female user at least twice per week or three, four, five, six times per week or more for a period of at least 4 weeks or 8 weeks or 12 weeks or more. In other instances, the method may be performed daily for a period of at least 4 weeks or 8 weeks or 12 weeks or more. In some instances, the female user may perform the method for 6, 8, 10, 12 months or more. The method may be performed by a female user at about the same time each day or following a daily ritual activity in order to facilitate habit adoption and habit compliance. For example, the female user may perform the method in the morning, as part of a routine (e.g., after showering). Similarly, a female user may perform the method in the evening before bed or at any other time convenient to the female user. The applicator may be sufficiently durable and/or hygienic so that the female user may safely repeat the method 2, 5, 10, 20, 30, 40, or 50 times or more using the same applicator. While it is desirable for the applicator to be durable, it is appreciated that daily use may require replacement of the applicator by a new or fresh applicator after 2, 3, 4 weeks or more of use. The new or fresh applicator may be obtained separately or as part of a new kit that includes a new applicator. The female user may use from about 6, 12, 18 or more fresh or new kits per year. The applicator, protective container and dispenser may be travel sized so that they can be stored in a purse if desired. The applicator may be disposable or intended for a single use.

### E. Test Procedures

i. Absorbency

Absorbency is measured as the gravimetric fluid uptake of an applicator sample submerged into a test fluid. Prepare a 0.042% w/v of Tritan X-100 solution, (reagent grade Sigma-Aldrich, or equivalent) in purified water (e.g. water from a Mill-iQ system) as the test fluid. The fluid's surface tension is tested and the result should be 32 dyne±1 dyne. All testing is performed in a conditioned room maintained at about 23° C.±2° C. and about 50%±2% relative humidity and samples are conditioned under these conditions for 2 hours before testing.

Measure the mass of the applicator and record as the Dry Mass to the nearest 0.0001 g. Place 400 mL of test fluid into a 500 mL beaker. If needed, the fluid level and beaker can be chosen such that the applicator can be completely submerged without overflowing the beaker. Soak the sample for 5.0 min±0.1 min. With tongs remove the sample and place onto a wire rack with the tip directed downward. After 1.0 min±0.1 min, place the sample into a tared weigh boat and measure the mass of the sample. Record the Wet Mass to the nearest 0.0001 g. The Absorbency (%) is calculated as (Wet Mass-Dry Mass)/Dry Mass and recorded to the nearest 0.01%. Repeat the measure for a total of five samples. Calculate the arithmetic average of the five results and report to the nearest 0.01%.

ii. Viscosity at 25° C.

The viscosity of samples is measured using Brookfield RV viscometer fitted with a heliopath T-bar spindle type appropriate for the viscosity being measured. The viscometer is leveled, setup and calibrated according to the manufacturer's standards. The viscometer speed (RPM) is selected to ensure the measured viscosity is within the manufactures recommended settings.

Samples are stored in sealed glass jars with an opening and internal diameter of at least 40 mm and filled to a height of at least 50 mm with care taken to avoid entrapped air bubbles. Centrifugation may be used to help remove entrained air. Sample jars are equilibrated at 25° C.±2° C. and about 50%±2% relative humidity for at least 24 hours prior to measurement.

Viscosity is measured at 25° C.±2° C. and about 50%±2% relative humidity by placing the uncapped sample jar under the viscometer and lowering the viscometer until the tip of the t-bar touches the surface of the sample. The descending heliopath is turned on and a timer started once the cross-bar of the t-bar touches the surface of the sample. For 1 minute, a reading is taken every 10 seconds. The viscosity is the arithmetic average of the viscosities recorded. Care is taken to ensure the t-bar does not touch the glass jar.

iii. Viscosity at 70° C.

The viscosity of samples is measured using Brookfield RV viscometer fitted with a Brookfield spindle appropriate for the viscosity being measured. The viscometer is leveled, setup and calibrated according to the manufacturer's standards. The viscometer speed (RPM) is selected to ensure the measured viscosity is within the manufactures recommended settings.

Samples are stored in sealed glass jars with an opening and internal diameter of at least 40 mm and filled to a height of at least 50 mm with care taken to avoid entrapped air bubbles. Centrifugation may be used to help remove entrained air. Sample jars are equilibrated at 70° C.±2° C. and about 50%±2% relative humidity for at least 24 hours prior to measurement.

Viscosity is measured at 70° C.±2° C. and about 50%±2% relative humidity by placing the uncapped sample jar under the viscometer and lowering the viscometer until the fluid touches the indication line. The viscometer is turned on and a reading is taken every 10 seconds for 1 minute. The viscosity is the arithmetic average of the viscosities recorded. Care is taken to ensure the spindle does not touch the glass jar.

### Examples

For examples 1-2, in a suitable container, combine the ingredients of Phase A. In a separate suitable container, combine the ingredients of Phase B. Heat each phase to 73-78° C. while mixing each phase using a suitable mixer (e.g., Anchor blade, propeller blade, or IKA T25) until each reaches a substantially constant desired temperature and is homogenous. Slowly add Phase B to Phase A while continuing to mix Phase A. Continue mixing until batch is uniform. Pour product into suitable containers at 73-78° C. and store at room temperature. Alternatively, continuing to stir the mixture as temperature decreases results in lower observed hardness values at 21° C. and 33° C. For examples 3-4, in a suitable container, combine the ingredients of Phase B and heat to 90° C. In a separate suitable container, combine the ingredients of Phase A and heat to approximately 80-85° C. until the components melt. In a third container, combine the ingredients of Phase C. Rapidly add Phase C to Phase A, while continuing to heat and stir the mixture. Pour phase B into the mixture of Phases A and C and stir. Pour the hot product into room temperature dishes, fit with closure, and allow to cool to room temperature.

| | 1 | 2 | 3 | 4 |
|---|---|---|---|---|
| PHASE A | | | | |
| DC-9040¹ | 5.12 | 5.00 | | |
| Dimethicone | 4.09 | 4.00 | | |
| Polymethylsilsesquioxane² | 4.09 | 4.00 | | |
| Cyclomethicone | 11.43 | 11.33 | | |
| KSG-210³ | 5.37 | 5.40 | 4.82 | |
| Polyethylene wax⁴ | 2.05 | 2.05 | 0.80 | 0.80 |
| DC-2503 Cosmetic Wax⁵ | 3.77 | 3.77 | | |
| Crodamol ™ CAP⁶ | | | 2.44 | 2.64 |
| Purcell ™ Jojoba Esters 70⁷ | | | 5.50 | 5.50 |
| Abil ™ WE09⁸ | | | | 1.92 |
| Hydrophobic Ti02 | 0.49 | 0.50 | | |
| Iron oxide coated Mica | | 0.65 | | |
| Fragrance | 0.10 | 0.10 | | |
| PHASE B | | | | |
| Glycerin | 10.00 | 10.00 | 2.00 | 2.00 |
| Dexpanthenol | 0.50 | 0.50 | | |
| Pentylene glycol | 3.00 | 3.00 | | |
| Hexamidine | 0.10 | 0.10 | | |
| Diisethionate⁹ | | | | |
| Niacinamide | 5.00 | 5.00 | | |
| Methylparaben | 0.20 | 0.20 | | |
| Ethylparaben | 0.05 | 0.05 | | |
| Sodium citrate | 0.20 | 0.20 | | |
| Citric acid | 0.03 | 0.03 | | |
| Sodium benzoate | 0.05 | 0.05 | | |
| Sodium chloride | 0.50 | 0.50 | 0.50 | 0.50 |
| FD&C Red #40 (1%) | 0.05 | 0.05 | | |
| Water | q.s | q.s | q.s | q.s |
| | to 100 | to 100 | to 100 | to 100 |
| PHASE C | | | | |
| Crodamol ™ CAP⁶ | | | 2.00 | 2.00 |
| Cyclomethicone | | | 4.44 | 4.59 |

¹12.5% Dimethicone Crosspolymer in Cyclopentasiloxane. Available from Dow Coming ™.
²E.g., Tospearl ™ 145A or Tospearl 2000. Available from GE Toshiba Silicone ™.
³25% Dimethicone PEG-10/15 Crosspolymer in Dimethicone. Available from Shin-Etsu ™.
⁴Jeenate ™ 3H polyethylene wax from Jeen ™, incorporated into Examples 1-2. Performalene ™ 400 polyethylene wax from New Phase Technologies ™ incorporated into Examples 3-4.
⁵Stearyl Dimethicone. Available from Dow Corning.
⁶Cetearyl octanoate and isopropyl myristate. Available from Croda Inc. ™.
⁷Hydrogenated Jojoba Oil. Available from Purcell Jojoba International ™.
⁸Preparation of cetyl dimethicone copolyol, polyglyceryl-4-isostearate and hexyl laurate. Available from Goldschmidt Chemical ™.
⁹Hexamidine disethionate, availabile from Laboratoires Serobiologiques.

For examples 5-6, formulate by combining the solid thickening agents (gellants) and liquid carriers in a vessel equipped with a heat source. The combined solids and liquids are heated to a temperature ranging from 85° C. to 96° C. and agitated to dissolve the solid thickening agents until the mixture forms a homogeneous clear to slightly cloudy solution, at which point any solid cosmetic active is added to and dispersed throughout the heated solution while maintaining mixing. The complexed or encapsulated compounds are then added to the melted mixture. If using a cyclodextrin complex, it is preferable to use a cyclodextrin complex with a degree of complexing greater than about 75% to prevent perfume from flashing or evaporating in the heating stage of the making process and to prevent compounds from the complex from blending in with free perfume added separately. If needed, the mixture may then be milled to eliminate solid particles or clumps and render the composition smooth and free of large particles. Finally, an optional perfume may be added at this stage, just prior to or after cooling. The resulting heated combination is then circulated through a scraped wall heat exchanger and cooled to 62° C. before filling the cooled mixture into plastic dispensing canisters and allowed to cool and solidify within the canisters over a 20 minute period (cooling rate of 2° C./min) through a forced air cooling tunnel having an air temperature of 21° C.

| | 5 | 6 |
|---|---|---|
| Ozokerite wax | 16.00 | 17.00 |
| Cyclopentasiloxane | 46.81 | 46.81 |
| petrolatum | 4.00 | 5.00 |
| Spray dried beta cyclodextrin | 5.00 | 5.00 |
| Perfume Complex (95% Degree of Complexing) | | |
| Zinc oxide | 5.00 | |
| Dimethicone 50 cst | 3.00 | 3.00 |
| C12-C15 Alkyl Benzoate | 10.00 | 10.00 |
| PPG 14. Butyl Ether (Fluid AP) | 10.00 | 10.00 |
| Behenyl Alcohol | 0.19 | 0.19 |
| Panthenyl Triacetate | | 3.00 |
| Totals | 100.00 | 100.00 |

For example 7, phase 1 (the caprylic/capric triglycerides) is first heated to 90° C. and mixing begins with the use of a vortex creating mixer. Phases 2, 3, 4, 5, 6, and 7 are added (preferably) sequentially and cooling to 60° C. is allowed during mixing. The batch can then be cooled to room temperature.

| | | 7 |
|---|---|---|
| Phase 1 | Caprylic/capric triglycerides | 51.40 |
| Phase 2 | Allantoin QA | 0.50 |
| Phase 3 | DL Alpha Tocopherol | 0.10 |
| Phase 4 | Ozokerite (Melt Pt. 75° C.)/117P/SP 1020 | 13.20 |
| Phase 5 | Compritol 888 ATO | 2.80 |
| Phase 6 | Cetearyl alcohol | 20.00 |
| Phase 7 | Stearyl alcohol | 12.00 |

### Combinations

A. A product for treating the vagina, preferably the vaginal introitus and/or external vaginal tissues, comprising i) an applicator comprising a receptacle comprising a first portion, a second portion, and an attachment structure, wherein the first portion and second portion are removably attached to define a substantially spherical shape, and ii) a vaginal care composition stored within the receptacle, preferably wherein the applicator is hand-held.
B. A product for treating the vagina, preferably the vaginal introitus and/or external vaginal tissues, comprising: i) a substantially spherical applicator, and ii) a vaginal care composition, wherein the applicator is shaped and configured to hold the vaginal care composition composition and the composition has a viscosity from about 10 cps to about 200,000 cps, preferably from about 15 cps to about 100,000 cps, more preferably from about 20 cps to about 50,000 cps, even more preferably from about 25 cps to about 40,000 cps when measured at 70° C., wherein the applicator is preferably hand-held.
C. A method for treating the vagina, preferably the vaginal introitus and/or external vaginal tissues, comprising: i) applying a vaginal care composition from a dispenser storing the vaginal care composition to a substantially spherical, solid applicator, ii) subsequently inserting the applicator into the vagina, wherein the vagina is treated substantially without digital application of the vaginal composition to the vagina.
D. The product or method according to any one of the preceding paragraphs, wherein the product treats vaginal dryness, vaginal irritation, vaginal itch, vaginal infection, vaginal odor, vaginal chafing, dyspareunia, and/or vaginal atrophy.
E. The product or method according to any one of the preceding paragraphs, wherein the applicator has an overall length L1 from about 20 mm to 90 mm, preferably from about 30 mm to about 80 mm, more preferably from about 40 mm to 70 mm; and a maximum width Wmax from about 20 mm to about 80 mm, preferably from about 25 mm to about 70 mm, more preferably from about 30 mm to about 50 mm.
F. The product or method according to any one of the preceding paragraphs, wherein the vaginal care composition has a viscosity from about 10 cps to about 200,000 cps, preferably from about 15 cps to about 100,000 cps, more preferably from about 20 cps to about 50,000 cps, even more preferably from about 25 cps to about 40,000 cps when measured at 70° C. [symbol]
G. A method for treating the vagina, preferably the vaginal introitus and/or external vaginal tissues, comprising:
   a. providing a female user with the product of paragraph A;
   b. the female user grasping the applicator, detaching the first portion of the applicator from the second portion of the applicator, and applying the vaginal care composition, preferably an effective amount of the vaginal care composition, more preferably from about 0.1 g to about 2 g of the vaginal care composition, even more preferably from about 0.5 g to about 1 g of the vaginal care composition, most preferably about 1 g of the vaginal care composition, to her vaginal introitus and, optionally, an external vaginal tissue selected from the group consisting of the vulva, vestibule, labia majora, labia minora, and external urogenital tract, and
      optionally reattaching the first portion of the applicator to the second portion of the applicator, preferably wherein the vagina is treated substantially without digital application of the vaginal composition to the vagina.
H. The method of paragraph C or paragraph G, wherein the female user inserts the applicator into the vagina a distance of about 35 mm or less, preferably 30 mm or less, more preferably 25 mm or less.
I. The product or method according to any one of the preceding paragraphs, wherein the applicator comprises a thermoplastic, a polyurethane, a thermoplastic elastomer, a cross-linked silicone rubber, or a combination thereof, optionally the polyurethane, thermoplastic elastomer or cross-linked silicone rubber.
J. The product or method according to any one of the preceding paragraphs, wherein the applicator comprises a cross-linked silicone rubber, preferably a medical grade cross-linked silicone rubber.
K. The product or method according to any one of the preceding paragraphs, wherein the vaginal composition is smooth.
L. The product or method according to any one of the preceding paragraphs, wherein the vaginal care composition is substantially free of an estrogen agent, a progesterone agent, or a combination thereof.
M. The product or method according to any one of the preceding paragraphs wherein the applicator is substantially free or devoid of moving parts.
N. The product or method according to any one of the preceding paragraphs, wherein the applicator is non-electrical.
O. The product or method according to any one of the preceding paragraphs, wherein the applicator comprises a grippable portion comprising a texture, optionally the texture is disposed adjacent to the bottom, the texture partially or wholly encircles the body, the texture has a maximum height or a maximum depth from about 0.1 mm to about 4 mm, or about 0.25 mm to about 3 mm, or about 1 mm to about 2 mm, the texture is in the form of a plurality of ridges, a plurality of grooves, a plurality of dimples, a plurality of flowers or a plurality of leaves, or the texture is provided as a repeating pattern.
P. The product or method according to any one of the preceding paragraphs, wherein the vaginal care composition is a solid or a semi-solid, preferably having a surface area from about 500 mm2 to about 4,000 mm2, or about 700 mm2 to about 3,000 mm2, or about 700 mm2 to about 2,000 mm2.
Q. The product or method according to any one of the preceding paragraphs, wherein the cross-sectional shape of the applicator at the base of the first portion is circular or ovoidal.
R. The product or method according to any one of the preceding paragraphs, wherein the applicator is not phallic shaped.
S. The product or method according to any one of the preceding paragraphs, wherein the bottom further comprises a substantially planar portion and wherein the applicator preferably stands upright when placed on flat surface.
T. The product or method according to any one of the preceding paragraphs, wherein the applicator has a rotational symmetry about its longitudinal axis thru a rotation of 45°, 90°, 135°, 180°, 225°, 270°, 315° or 360°.
U. The product or method according to any one of the preceding paragraphs, wherein the first portion has a length L2 from about 10 mm to about 40 mm.
V. The product or method according to any one of the preceding paragraphs, wherein a ratio of the overall length L1 of the applicator to the maximum width Wₘₐₓ of the applicator is from about 1:2 to about 1:1.
W. The product or method according to any one of the preceding paragraphs, wherein the applicator tapers from the maximum width Wₘₐₓ to the tip and the applicator tapers from the maximum width Wₘₐₓ to the bottom.
X. The product or method according to any one of the preceding paragraphs, wherein the vaginal care composition comprises i) petrolatum, beeswax, carnauba wax, coconut oil, olive oil, and/or jojoba oil, and ii) an ingredient selected from the group consisting of emollients, moisturizers, vitamins, aloe vera, colorants, vitamins, fragrances, and mixtures thereof.
Y. The product or method according to any one of the preceding paragraphs, wherein the lower portion and the upper portion each include a cover layer of thermoplastic elastomer.
Z. The product or method according to any one of the preceding paragraphs, wherein the lower portion and the upper portion comprise acrylonitrile-butadiene-styrene copolymer.
AA. The product or method according to any one of the preceding paragraphs, wherein the lower portion and the upper portion comprise polyethylene.
BB. The product or method according to any one of the preceding paragraphs, wherein the vaginal care composition is substantially free of one or more of the following: retinol, retinyl esters, retinaldehyde, peptides, ethanol, sunscreens or ultraviolet protective agents, sensates, perfumes, colorants, dyes, pigments, and particulates having an average particle size greater than 125 microns.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A product for treating the vagina comprising i) a hand-held applicator comprising a receptacle comprising a first portion, a second portion, and an attachment structure, wherein the first portion and second portion are removably attached to define a substantially spherical shape, and ii) a vaginal care composition stored within the receptacle.

2. A product for treating the vagina comprising: i) a substantially spherical, hand-held applicator, and ii) a vaginal care composition, wherein the applicator is shaped and configured to hold the vaginal care composition and the composition has a viscosity from about 10 cps to about 200,000 cps when measured at 70° C.

3. A method for treating the vagina comprising: i) applying a vaginal care composition from a dispenser storing the vaginal care composition to a substantially spherical, solid applicator, ii) subsequently inserting the applicator into the vagina, wherein the vagina is treated substantially without digital application of the vaginal composition to the vagina.

4. The product of claim 1, wherein the product treats vaginal dryness, vaginal irritation, vaginal itch, vaginal infection, vaginal odor, vaginal chafing, dyspareunia, and/or vaginal atrophy.

5. The product of claim 1, wherein the applicator has an overall length L1 from about 20 mm to 90 mm and a maximum width Wmax from about 20 mm to about 80 mm.

6. The product of claim 1, wherein the vaginal care composition has a viscosity from about 10 cps to about 200,000 cps when measured at 70° C.

7. A method for treating the vagina comprising:
a. providing a female user with the product of claim 1;
b. the female user grasping the applicator, detaching the first portion of the applicator from the second portion of the applicator, and applying the vaginal care composition to her vaginal introitus.

8. The method of claim 3, wherein the female user inserts the applicator into the vagina a distance of about 35 mm or less.

9. The product of claim 1, wherein the applicator comprises a thermoplastic, a polyurethane, a thermoplastic elastomer, a cross-linked silicone rubber, or a combination thereof.

10. The product of claim 1, wherein the vaginal care composition is substantially free of an estrogen agent, a progesterone agent, or a combination thereof.

11. The product of claim 1, wherein the applicator is substantially free or devoid of moving parts.

12. The product of claim 1, wherein the applicator is non-electrical.

13. The product of claim 1, wherein the applicator comprises a grippable portion comprising a texture.

14. The product of claim 1, wherein the vaginal care composition is a solid or a semi-solid having a surface area from about 500 mm2 to about 4,000 mm2.

15. The product of claim 1, wherein the applicator is not phallic shaped.

16. The product of claim 1, wherein the applicator further comprises a substantially planar portion and the applicator stands upright when placed on a flat surface.

17. The product of claim 1, wherein the applicator has a rotational symmetry about its longitudinal axis thru a rotation of 45°.

18. The product of claim 1, wherein the first portion has a length L2 from about 10 mm to about 40 mm.

19. The product of claim 1, wherein the vaginal care composition comprises i) petrolatum, beeswax, carnauba wax, coconut oil, olive oil, jojoba oil, or a mixture thereof and ii) an ingredient selected from the group consisting of emollients, moisturizers, vitamins, aloe vera, colorants, vitamins, fragrances, and mixtures thereof.

20. The product of claim 1, wherein the vaginal care composition is substantially free of one or more of the following: retinol, retinyl esters, retinaldehyde, peptides, ethanol, sensates, perfumes, colorants, dyes, pigments, and particulates having an average particle size greater than 125 microns.
